(19) United States
(12) Patent Application Publication
(10) Pub. No.: US 2021/0395394 A1
(43) Pub. Date: Dec. 23, 2021

(54) Antibody against pan-species-specific plasmodium lactate dehydrogenase

(71) Applicant: Fapon Biotech Inc., Shenzhen,Guangdong (CN)

(72) Inventors: Peng CUI, Dongguan, Guangdong (CN); Zhiqiang HE, Dongguan, Guangdong (CN); Yuan MENG, Dongguan, Guangdong (CN); Dongmei ZHONG, Dongguan, Guangdong (CN); Lina TANG, Dongguan, Guangdong (CN); Bi LIANG, Dongguan, Guangdong (CN); Hui YOU, Dongguan, Guangdong (CN)

(21) Appl. No.: 17/293,046

(22) PCT Filed: Oct. 1, 2019
(86) PCT No.: PCT/CN2019/109790

§ 371 Date: May 12, 2021

(30) Foreign Application Priority Data
Dec. 25, 2018 (CN) 201811595399.7

(51) Int. Cl. C07K 16/40; G01N 33/569

(52) U.S. Cl. CPC C07K 16/40; G01N 33/569; G01N 2800/26; C07K 2317/92; C07K 2317/52; C07K 2317/565

(57) ABSTRACT

Provided are an isolated binding protein including a pan-species-specific plasmodium lactate dehydrogenase antigen binding domain and a preparation method thereof. The antigen binding domain includes at least one complementarity determining region selected from a defined amino acid sequence, or has at least 80% of sequence identity with the complementarity determining region of the following amino acid sequence and an affinity of K_{D}≤1.5647×10⁻⁹ mol/L with a pan-species-specific plasmodium lactate dehydrogenase, and may identify the pan-species-specific plasmodium lactate dehydrogenase. The binding protein may be applied to the field of detection of plasmodium lactate dehydrogenase proteins.

### CROSS-REFERENCE TO RELATED APPLICATION

The present application claims priority to Chinese Patent Application No. 201811595399.7, filed to the China National Intellectual Property Administration on Dec. 25, 2018 and entitled “Antibody against pan-species-specific plasmodium lactate dehydrogenase”, the disclosure of which is hereby incorporated by reference in its entirety.

### TECHNICAL FIELD

The present disclosure relates to the field of immunological technologies, and in particular to an antibody against a pan-species-specific plasmodium lactate dehydrogenase.

### BACKGROUND

Malaria is an insect-borne infectious disease caused by infection of a plasmodium by bite of anopheles or transfusion of blood of a person carrying the plasmodium. There are four main types of the plasmodium parasitizing in a human body: Plasmodium vivax (Pv), Plasmodium falciparum (Pf), Plasmodium malariae (Pm), and Plasmodium ovale (Po). The Plasmodium falciparum is a common infectious plasmodium (75%), and is the most harmful pathogen. It has strong infectivity, rapid proliferation, and severe symptoms. A mortality rate of the primary infection is high, deaths caused by it account for more than 95% of a total deaths of infected persons, and it mainly exists in tropical areas of Africa, South America and Asia. The Plasmodium vivax is the second most common plasmodium (20%), and is also a most common type in areas outside Africa. The World Health Organization (WHO) recommends that all suspected malaria patients should be detected for the malaria immediately. Rapid and accurate diagnosis of the disease is essential for correctly using an antimalarial drug, avoiding generation of a drug-resistant strain, controlling deterioration of the disease, and reducing the mortality rate.

Malaria diagnosis is a key point of malaria control. Classified by a detection technology principle, At present, the methods for detecting the plasmodium may be divided into four types. The first type is to directly detect the plasmodium with a microscope, including a thick blood membrane and a thin blood membrane, this is also a gold standard for clinical diagnosis of the malaria at present. However, it is time-consuming and labor-consuming, and requires a skilled technician and a certain experiment condition. The second type is plasmodium nucleic acid detection, a detection target is a specific nucleotide fragment such as a plasmodium 18S ribosomal RNA, and commonly used methods are a fluorescent PCR method and a loop-mediated isothermal amplification (LAMP) technology. Although this type of the methods has high sensitivity and specificity, more complicated instruments and technical conditions are required as support, it is not suitably used as a conventional detection method in malaria-endemic areas, and is difficult to promote and apply at a grassroots level. The third type is to detect a pigment of the plasmodium, and a flow cytometry or a mass spectrometry is usually used. This method requires a professional detection instrument, and is generally used for a laboratory research, and is not suitable for on-site detection. The fourth type is to detect the plasmodium by an antigen-antibody immune response, there are an immunochromatographic Rapid Diagnostic Reagent (RDT) and an Enzyme-Linked ImmunoSorbent Assay (ELISA) methodologically, and most of target antigens detected are diagnostic antigens such as a Plasmodium Lactate DeHydrogenase (PLDH) and a Histidine-Rich Protein II (HRP-II). The RDT using the antigen as a detection target has important significance while being applied in backward areas where the Plasmodium falciparum is prevalent. It is recommended by the WHO for on-site diagnosis due to advantages of its simple operation, rapidness, intuitive results, no complicated device, high sensitivity and specificity and the like.

At present, the malaria antigens commonly used in the RDT method are mainly the Histidine-Rich Protein II (HRP-II) unique to the Plasmodium falciparum and the PLDH. The HRP-II is a specific antigen of the Plasmodium falciparum, and a most commonly used target antigen in detection of falciparum malaria. The Plasmodium Lactate DeHydrogenase (PLDH) is an important enzyme to guarantee the normal progress of plasmodium glycolysis. Compared with lactate dehydrogenases of human erythrocytes and many other microorganisms, it has significantly different physical and biochemical properties. It is a protein that must be expressed during a life activity process of the plasmodium, and has a higher abundance, so it becomes an important target for detection of the plasmodium. Since the PLDH is only produced by the live plasmodium, a method of using the PLDH as a detection antigen may also identify life and death of the plasmodium in a patient, thereby the therapeutic effect and recurrence situation may be evaluated and monitored. In addition, the PLDH produced by the four types of the plasmodium has different isomers, such as species and genus-specific antigens, it may be mainly divided into two categories: the first is a species-specific LDH, including pfLDH, pvLDH, pmLDH, and poLDH, a monoclonal antibody produced with this as a target protein only recognizes the LDH of the specific species of the plasmodium; and the second is a Pan-species specific antigen Plasmodium Lactate DeHydrogenase (Pan-PLDH), a monoclonal antibody produced with this as a target protein may recognize the LDH of four types of the plasmodium.

At present, kits on the market that use the Pan-PLDH as the target protein to detect the plasmodium mainly include a CareStart malaria HRP-II/PLDH composite test kit and an OptiMAL diagnostic kit. The CareStart malaria HRP-II/PLDH composite test kit is produced by Access Bio Company, USA. It uses two monoclonal antibodies to form two independent detection lines on a membrane, and they are respectively an anti-plasmodium (Plasmodium falciparum, Plasmodium vivax, Plasmodium ovale and Plasmodium malariae) lactate dehydrogenase (Pan-PLDH) monoclonal antibody and an anti-HRP-II monoclonal antibody, and are specially used for differential diagnosis of falciparum malaria and other types of the malaria. The OptiMAL diagnostic kit is produced in Oregon, Poland. A chromatogram strip is coated with two strains of plasmodium LDH monoclonal antibodies, one strain is a Plasmodium falciparum species-specific monoclonal antibody, and the other strain is a genus-specific monoclonal antibody that may react with all of the four types of the human plasmodium, so it may distinguish Plasmodium falciparum or non-Plasmodium falciparum infection. In addition, similar kits include a NovaBios plasmodium antigen (pf/pan-PLDH) test kit of US, an SD Plasmodium vivax antigen test kit P.f/Pan (HRP-2/pLDH) of South Korea, and a Binax NOW malaria rapid test kit of US, and a Wanfu plasmodium detection kit (Pf-LDH/Pan-PLDH), etc.

In the above kits, anti-Pan-PLDH monoclonal antibodies are used. At present, a conventional preparation method of the monoclonal antibodies used for diagnosis in the market is a hybridoma technology, namely, a genetic engineering technology is used to express a Pan-species-specific antigen Plasmodium Lactate DeHydrogenase (Pan-PLDH) protein to immunize a mouse, spleen cells of the immunized mouse are fused with tumor cells to obtain hybridoma cells, and finally, a monoclone that secretes the target antibody is screened out from the hybridoma cells, and then the antibody is provided. So far, the traditional hybridoma technology is still one of the main methods for preparing the monoclonal antibodies due to its low cost, sustainable production, good operability and advantages in clinical diagnosis. However, with the traditional hybridoma technology, during processes of culture or cryopreservation and recovery of the hybridoma cells, some cells may lose an ability to secrete the antibodies, so that some precious cell lines are lost. In addition, while a large number of the antibodies are produced, a large number of the hybridoma cells are cultured in vitro, and a yield thereof is low. Generally, the antibody content in culture solution is 10-60 mg/L. If mass production is performed, the cost is higher; and in a mouse abdominal cavity induction process, due to the influence of a mouse individual size, the production of the antibodies is unstable, a difference between batches is large, and purification difficulty is large because mouse autoantibodies are contained.

In order to avoid disadvantages of the traditional hybridoma technology, the present disclosure designs an expression vector for a monoclonal antibody against a Pan-species-specific antigen Plasmodium Lactate DeHydrogenase (Pan-PLDH), and provides a monoclonal antibody sequence against the Pan-species-specific antigen Plasmodium Lactate DeHydrogenase (Pan-PLDH), a host cell for expressing the monoclonal antibody against the Pan-species-specific antigen Plasmodium Lactate DeHydrogenase (Pan-PLDH) by a recombinant technology, and a diagnostic method for the malaria.

### SUMMARY

The present disclosure relates to a novel isolated binding protein containing a pan-species-specific antigen plasmodium lactate dehydrogenase antigen binding domain, and researches the binding protein in aspects of preparation, application and the like.

Herein the antigen binding domain includes at least one complementarity determining region selected from the following amino acid sequences; or has at least 80% of sequence identity with the complementarity determining region of the following amino acid sequences and an affinity of K_{D}≤1.5647×10⁻⁹ mol/L with a pan-species-specific plasmodium lactate dehydrogenase;

a complementarity determining region CDR-VH1 is G-X1-S-F-T-N-Y-X2-M-N, herein

X1 is S, Y or T, and X2 is W or F;

a complementarity determining region CDR-VH2 is I-X1-P-S-X2-S-E-T-R-X3-N-Q, herein

X1 is H or N, X2 is E or D, and X3 is I, V or L;

a complementarity determining region CDR-VH3 is A-X1-S-G-X2-F-Y-T-X3-Y-X4-D-Y, herein,

X1 is K or R, X2 is D or E, X3 is S, Y or T, and X4 is F or W;

a complementarity determining region CDR-VL1 is R-G-X1-G-N-X2-H-N-Y-X3-A, herein,

X1 is S or T, X2 is I, V or L, and X3 is I or L;

a complementarity determining region CDR-VL2 is N-A-X1-T-X2-A-D, herein

X1 is R or K, and X2 is I, V or L;

a complementarity determining region CDR-VL3 is Q-X1-F-W-S-X2-Y-T, herein

X1 is S, Y or T, and X2 is S or T.

An important advantage is that the binding protein has strong activity and high affinity with the pan-species-specific antigen plasmodium lactate dehydrogenase.

In one or more implementation modes:

in the complementarity determining region CDR-VH1, the X2 is W;

in the complementarity determining region CDR-VH2, the X1 is H;

in the complementarity determining region CDR-VH3, the X4 is F;

in the complementarity determining region CDR-VL1, the X3 is L; and

in the complementarity determining region CDR-VL3, the X2 is T.

In one or more implementation modes, in the complementarity determining region CDR-VH1, the X1 is S.

In one or more implementation modes, in the complementarity determining region CDR-VH1, the X1 is Y.

In one or more implementation modes, in the complementarity determining region CDR-VH1, the X1 is T.

In one or more implementation modes, in the complementarity determining region CDR-VH2, the X2 is E.

In one or more implementation modes, in the complementarity determining region CDR-VH2, the X2 is D.

In one or more implementation modes, in the complementarity determining region CDR-VH2, the X3 is I.

In one or more implementation modes, in the complementarity determining region CDR-VH2, the X3 is V.

In one or more implementation modes, in the complementarity determining region CDR-VH2, the X3 is L.

In one or more implementation modes, in the complementarity determining region CDR-VH3, the X1 is K.

In one or more implementation modes, in the complementarity determining region CDR-VH3, the X1 is R.

In one or more implementation modes, in the complementarity determining region CDR-VH3, the X2 is D.

In one or more implementation modes, in the complementarity determining region CDR-VH3, the X2 is E.

In one or more implementation modes, in the complementarity determining region CDR-VH3, the X3 is S.

In one or more implementation modes, in the complementarity determining region CDR-VH3, the X3 is Y.

In one or more implementation modes, in the complementarity determining region CDR-VH3, the X3 is T.

In one or more implementation modes, in the complementarity determining region CDR-VL1, the X1 is S.

In one or more implementation modes, in the complementarity determining region CDR-VL1, the X1 is T.

In one or more implementation modes, in the complementarity determining region CDR-VL1, the X2 is I.

In one or more implementation modes, in the complementarity determining region CDR-VL1, the X2 is V.

In one or more implementation modes, in the complementarity determining region CDR-VL1, the X2 is L.

In one or more implementation modes, in the complementarity determining region CDR-VL2, the X1 is R.

In one or more implementation modes, in the complementarity determining region CDR-VL2, the X1 is K.

In one or more implementation modes, in the complementarity determining region CDR-VL2, the X2 is I.

In one or more implementation modes, in the complementarity determining region CDR-VL2, the X2 is V.

In one or more implementation modes, in the complementarity determining region CDR-VL2, the X2 is L.

In one or more implementation modes, in the complementarity determining region CDR-VL3, the X1 is S.

In one or more implementation modes, in the complementarity determining region CDR-VL3, the X1 is Y.

In one or more implementation modes, in the complementarity determining region CDR-VL3, the X1 is T.

In one or more implementation modes, mutation sites of each complementarity determining region are selected from any one of the following mutation combinations:

| | CDR-VH1 | CDR-VH2 | CDR-VH3 | CDR-VL1 | CDR-VL2 | CDR-VL3 |
|---|---|---|---|---|---|---|
| Site | X1 | X2/X3 | X1/X2/X3 | X1/X2 | X1/X2 | X1 |
| Mutation | S | E/I | K/D/S | S/L | R/I | S |
| combination 1 | | | | | | |
| Mutation | Y | E/L | K/D/Y | T/L | R/V | Y |
| combination 2 | | | | | | |
| Mutation | T | E/V | K/D/T | S/V | R/L | T |
| combination 3 | | | | | | |
| Mutation | T | D/I | K/E/S | T/V | K/I | Y |
| combination 4 | | | | | | |
| Mutation | Y | D/L | K/E/Y | S/I | K/V | T |
| combination 5 | | | | | | |
| Mutation | S | D/V | K/E/T | T/I | K/L | S |
| combination 6 | | | | | | |
| Mutation | T | D/I | R/D/S | T/I | K/L | T |
| combination 7 | | | | | | |
| Mutation | S | D/L | R/D/Y | S/I | K/V | S |
| combination 8 | | | | | | |
| Mutation | Y | D/V | R/D/T | T/V | K/I | Y |
| combination 9 | | | | | | |
| Mutation | S | E/I | R/E/S | S/V | R/L | S |
| combination 10 | | | | | | |
| Mutation | T | E/L | R/E/Y | T/L | R/V | Y |
| combination 11 | | | | | | |
| Mutation | Y | E/V | R/E/T | S/L | R/I | T |
| combination 12 | | | | | | |
| Mutation | Y | D/I | K/D/S | S/L | K/V | Y |
| combination 13 | | | | | | |
| Mutation | T | D/L | K/D/Y | T/L | K/L | T |
| combination 14 | | | | | | |
| Mutation | S | D/V | K/D/T | S/V | K/I | S |
| combination 15 | | | | | | |
| Mutation | S | E/I | K/E/S | T/V | R/V | T |
| combination 16 | | | | | | |
| Mutation | Y | E/L | K/E/Y | S/I | R/L | S |
| combination 17 | | | | | | |
| Mutation | T | E/V | K/E/T | T/I | R/V | T |
| combination 18 | | | | | | |
| Mutation | T | D/I | R/D/S | T/I | K/I | S |
| combination 19 | | | | | | |
| Mutation | Y | D/L | R/D/Y | S/I | K/L | Y |
| combination 20 | | | | | | |
| Mutation | S | D/V | R/D/T | T/V | KV | T |
| combination 21 | | | | | | |
| Mutation | T | E/I | R/E/S | S/V | R/I | Y |
| combination 22 | | | | | | |
| Mutation | S | E/L | R/E/Y | T/L | R/I | T |
| combination 23 | | | | | | |
| Mutation | Y | E/V | R/E/T | S/L | R/V | S |
| combination 24 | | | | | | |
| Mutation | S | D/I | K/D/S | S/L | K/L | T |
| combination 25 | | | | | | |
| Mutation | T | D/L | K/D/Y | T/L | K/I | S |
| combination 26 | | | | | | |
| Mutation | Y | D/V | K/D/T | S/V | K/V | Y |
| combination 27 | | | | | | |
| Mutation | Y | E/I | K/E/S | T/V | R/L | S |
| combination 28 | | | | | | |
| Mutation | T | E/L | K/E/Y | S/I | R/I | Y |
| combination 29 | | | | | | |
| Mutation | S | E/V | K/E/T | T/I | R/V | T |
| combination 30 | | | | | | |
| Mutation | S | D/I | R/D/S | T/I | K/L | Y |
| combination 31 | | | | | | |
| Mutation | Y | D/L | R/D/Y | S/I | K/V | T |
| combination 32 | | | | | | |
| Mutation | T | D/V | R/D/T | T/V | K/I | S |
| combination 33 | | | | | | |
| Mutation | T | E/I | R/E/S | S/V | R/L | T |
| combination 34 | | | | | | |
| Mutation | Y | E/L | R/E/Y | T/L | R/V | S |
| combination 35 | | | | | | |
| Mutation | S | E/V | R/E/T | S/L | R/I | Y |
| combination 36 | | | | | | |
| Mutation | T | D/I | K/D/S | S/L | K/V | S |
| combination 37 | | | | | | |
| Mutation | S | D/L | K/D/Y | T/L | K/I | Y |
| combination 38 | | | | | | |
| Mutation | Y | D/V | K/D/T | S/V | K/L | T |
| combination 39 | | | | | | |
| Mutation | S | E/I | K/E/S | T/V | R/V | Y |
| combination 40 | | | | | | |
| Mutation | T | E/L | K/E/Y | S/I | R/I | T |
| combination 41 | | | | | | |
| Mutation | Y | E/V | K/E/T | T/I | R/L | S |
| combination 42 | | | | | | |
| Mutation | Y | D/I | R/D/S | T/I | K/I | T |
| combination 43 | | | | | | |
| Mutation | T | D/L | R/D/Y | S/I | K/V | S |
| combination 44 | | | | | | |
| Mutation | S | D/V | R/E/S | T/V | K/L | Y |
| combination 45 | | | | | | |
| Mutation | Y | E/I | R/E/Y | S/V | R/I | S |
| combination 46 | | | | | | |
| Mutation | T | E/L | R/E/T | T/L | R/V | Y |
| combination 47 | | | | | | |
| Mutation | T | E/V | K/D/S | S/L | R/L | T |
| combination 48 | | | | | | |
| Mutation | Y | D/I | K/D/Y | S/L | K/L | Y |
| combination 49 | | | | | | |
| Mutation | S | D/L | K/D/T | T/L | K/V | T |
| combination 50 | | | | | | |
| Mutation | T | D/V | K/E/S | S/V | K/I | S |
| combination 51 | | | | | | |
| Mutation | S | E/I | K/E/Y | T/V | R/L | T |
| combination 52 | | | | | | |
| Mutation | Y | E/L | K/E/T | S/I | R/V | S |
| combination 53 | | | | | | |
| Mutation | S | E/V | R/D/S | T/I | R/I | Y |
| combination 54 | | | | | | |
| Mutation | T | D/I | R/D/Y | T/I | K/V | S |
| combination 55 | | | | | | |
| Mutation | Y | D/L | R/D/T | S/I | K/I | Y |
| combination 56 | | | | | | |

In one or more implementation modes, the binding protein includes at least 3 CDRs; or, the binding protein includes at least 6 CDRs.

In one or more implementation modes, the binding protein is one of a nano-antibody, F(ab′)₂, Fab′, Fab, Fv, scFv, diabodies, and an antibody minimum recognition unit.

In one or more implementation modes, the binding protein includes sequences of light chain framework regions FR-L1, FR-L2, FR-L3 and FR-L4 successively shown in SEQ ID NO: 1-4, and/or sequences of heavy chain framework regions FR-H1, FR-H2, FR-H3 and FR-H4 successively shown in SEQ ID NO: 5-8.

In one or more implementation modes, the binding protein further includes an antibody constant region sequence.

In one or more implementation modes, the constant region sequence is selected from a sequence of a constant region of any one of IgG1, IgG2, IgG3, IgG4, IgA, IgM, IgE, and IgD;

In one or more implementation modes, the species source of the constant region is cattle, horse, dairy cow, pig, sheep, goat, rat, mouse, dog, cat, rabbit, camel, donkey, deer, mink, chicken, duck, goose, turkey, gamecock or human.

In one or more implementation modes, the constant region is derived from the mouse.

In one or more implementation modes, a light chain constant region sequence is shown in SEQ ID NO: 9.

In one or more implementation modes, a heavy chain constant region sequence is shown in SEQ ID NO: 10.

The present disclosure provides an isolated nucleic acid molecule, the nucleic acid molecule is DNA or RNA, and it encodes the binding protein of the present disclosure.

The present disclosure provides a vector, and it includes the nucleic acid molecule of the present disclosure.

The present disclosure provides a host cell, and it is transformed by the vector of the present disclosure.

The present disclosure provides a method for producing the binding protein of the present disclosure, herein it includes the following steps:

culturing the host cell of the present disclosure in a culture medium and under a suitable culture condition, and recovering the binding protein thus produced from the culture medium or from the cultured host cell.

The present disclosure provides an application of the binding protein described herein in preparing a diagnostic agent for diagnosing malaria.

The present disclosure provides a method for detecting a pan-species-specific antigen plasmodium lactate dehydrogenase in a test sample, and it includes:

a) under a condition sufficient for an antibody/antigen binding reaction to occur, contacting a pan-species-specific antigen plasmodium lactate dehydrogenase antigen with the binding protein of the present disclosure so as to form an immune complex; and

b) detecting the presence of the immune complex, the presence of the complex indicates the presence of the pan-species-specific antigen plasmodium lactate dehydrogenase in the test sample.

In one or more implementation modes, in the step a), the immune complex further includes a second antibody, and the second antibody binds to the binding protein.

In one or more implementation modes, in the step a), the immune complex further includes a second antibody, and the second antibody binds to the pan-species-specific antigen plasmodium lactate dehydrogenase.

The present disclosure provides a kit, and it includes the binding protein of the present disclosure.

The present disclosure further provides an application of the binding protein described herein in diagnosis of malaria.

The present disclosure further provides a method for diagnosing malaria, including:

A) under a condition sufficient for a binding reaction to occur, contacting a sample from a subject with the binding protein of the present disclosure so as to perform the binding reaction; and

B) detecting an immune complex produced by the binding reaction,

herein the presence of the immune complex indicates the presence of the malaria.

In one or more implementation modes, the method is based on a fluorescence immunoassay technology, a chemiluminescence technology, a colloidal gold immunoassay technology, a radioimmunoassay and/or an enzyme-linked immunoassay technology.

In one or more implementation modes, the sample is selected from at least one of whole blood, peripheral blood, serum or plasma.

In one or more implementation modes, the subject is a mammal, preferably a primate, more preferably a human.

In one or more implementation modes, the malaria is selected from a group consisting of Plasmodium vivax, Plasmodium falciparum, Plasmodium malariae, Plasmodium ovale or a combination thereof.

In one or more implementation modes, the malaria is a malaria caused by Plasmodium.

### BRIEF DESCRIPTION OF THE DRAWINGS

In order to more clearly describe specific implementation modes of the present disclosure or technical schemes in the prior art, drawings which are required to be used in the specific implementation modes or descriptions of the prior art are briefly introduced below. It is apparent that the drawings in the following descriptions are some implementation modes of the present disclosure, under a precondition without creative work, other drawings may also be acquired by those of ordinary skill in the art according to these drawings.

FIG. 1 is an electrophoresis diagram of a monoclonal antibody against a pan-species-specific antigen plasmodium lactate dehydrogenase in an embodiment of the present disclosure.

### DETAILED DESCRIPTION OF THE EMBODIMENTS

The present disclosure may be more easily understood through the following descriptions of some implementation schemes of the present disclosure and the detailed content of embodiments included therein.

Before the present disclosure is further described, it should be understood that the present disclosure may not be limited to the specific implementation schemes, because these implementation schemes are necessarily diverse. It should also be understood that terms used in the description are only to illustrate the specific implementation schemes, rather than as limitation, because a scope of the present disclosure may not only be defined in appended claims.

### Noun Definition

“Isolated binding protein including an antigen binding domain” generally refers to all proteins/protein fragments including the CDR regions. A term “antibody” includes polyclonal antibodies and monoclonal antibodies, as well as antigen compound binding fragments of these antibodies, including Fab, F(ab′)₂, Fd, Fv, scFv, diabodies and an antibody minimum recognition unit, as well as single-chain derivatives of these antibodies and fragments. The type of the antibody may be IgG1, IgG2, IgG3, IgG4, IgA, IgM, IgE, and IgD. In addition, the term “antibody” includes naturally-existing antibodies and non-naturally-existing antibodies, including, for example, chimeric, bifunctional and humanized antibodies, and related synthetic isoforms. The term “antibody” may be used interchangeably with “immunoglobulin”.

“Variable region” or “variable domain” of the antibody refers to an amino terminal domain of heavy chain or light chain of the antibody. The variable domain of the heavy chain may be referred to as “VH”. The variable domain of the light chain may be referred to as “VL”. These domains are usually the most variable part of the antibody and contain an antigen binding site. The light chain or heavy chain variable region (VL or VH) is formed by three called “complementarity determining region” or “CDR” and framework regions separating the three complementarity determining regions. Ranges of the framework regions and CDRs are precisely defined, for example in Kabat (see “Sequences of Proteins of Immunological Interest”, E. Kabat et al., U. S. Department of Health and Human Services, (1983)) and Chothia. The framework region of the antibody, namely the framework region of a combination of key component light chain and heavy chain, plays a role in positioning and aligning the CDRs, the CDRs are mainly responsible for binding to the antigen.

As used herein, “framework region”, “architecture region” or “FR” refers to regions of the antibody variable domain except those regions defined as the CDRs. Each antibody variable domain framework may be further subdivided into adjacent regions (FR1, FR2, FR3, and FR4) separated by the CDRs.

Usually, the variable regions VL/VH of the heavy chain and light chain may be obtained by arranging and connecting the following numbered CDRs and FRs in the following combination: FR1-CDR1-FR2-CDR2-FR3-CDR3-FR4.

As used herein, terms “purified” or “isolated” related with a polypeptide or a nucleic acid means that the polypeptide or the nucleic acid is not in its natural medium or in its natural form. Thus, the term “isolated” includes the polypeptide or the nucleic acid taken from its original environment, for example, if it is naturally existent, from the natural environment. For example, an isolated polypeptide generally does not contain at least some proteins or other cellular components that are normally bound to or usually mixed with or in solution. The isolated polypeptide includes the naturally existing polypeptide contained in a cell lysate, the polypeptide in purified or partially purified form, the recombinant polypeptide, the polypeptide expressed or secreted by cells, and the polypeptide in heterologous host cell or culture. Related with the nucleic acid, the term isolated or purified indicates, for example, that the nucleic acid is not in its natural genomic background (for example, in a vector, as an expression cassette, linked to a promoter, or artificially introduced into a heterologous host cell).

As used herein, a term “diabodies” or “bifunctional antibody” refers to an artificial hybrid binding protein with two different pairs of heavy/light chains and two different binding sites. A bispecific binding protein may be produced by a variety of methods, including fusion of hybridomas or linking of Fab′ fragments.

As used herein, a term “sequence identity” refers to similarity between at least two different sequences. This percentage identity may be determined by a standard algorithm, such as Basic Local Alignment Search Tool (BLAST); algorithms of Needleman and the like; or algorithms of Meyers and the like. In one or more implementation modes, a set of parameters may be a Blosum 62 scoring matrix and a gap penalty 12, a gap extension penalty 4, and a frameshift gap penalty 5. In one or more implementation modes, the percentage identity between two amino acid or nucleotide sequences may also be determined with an algorithm of Meyers and Miller ((1989) CABIOS 4:11-17), the algorithm has already incorporated in an ALIGN program (version 2.0), a PAM120 weight residue table, a gap length penalty 12, and a gap penalty 4 are used. The percentage identity is usually calculated by comparing sequences of a similar length.

As used herein, a term “affinity” refers to binding strength of the antigen binding domain of a binding protein or an antibody and an antigen or an epitope. The affinity may be measured by a KD value. The KD value is smaller, and the affinity is greater.

As used herein, terms “pan-species-specific antigen plasmodium lactate dehydrogenase” and “pan-species-specific plasmodium lactate dehydrogenase” may be used interchangeably, it means that the lactate dehydrogenase may be used as a pan-species-specific antigen of Plasmodium pathogens, for example, used as the pan-species-specific antigen of Plasmodium vivax (Pv), Plasmodium falciparum (Pf), Plasmodium malariae (Pm) and Plasmodium ovale (Po). In one or more implementation modes, the antibody against the pan-species-specific antigen plasmodium lactate dehydrogenase or its binding protein may specifically bind to or recognize LDH of the Plasmodium pathogens, thereby the malaria caused by the Plasmodium pathogens may be specifically diagnosed. In one or more implementation modes, the antibody against the pan-species-specific antigen plasmodium lactate dehydrogenase or its binding protein may bind to or recognize the LDH of Plasmodium vivax (Pv), Plasmodium falciparum (Pf), Plasmodium malariae (Pm), and Plasmodium ovale (Po), so that the malaria, including Plasmodium falciparum, Plasmodium vivax, Plasmodium ovale and Plasmodium malariae, caused by Plasmodium vivax (Pv), Plasmodium falciparum (P), Plasmodium malariae (Pm) and/or Plasmodium ovale (Po) is diagnosed.

### Exemplary Implementation Schemes of Present Disclosure

The present disclosure relates to an isolated binding protein containing an antigen binding domain, herein the antigen binding domain includes at least one complementarity determining region selected from the following amino acid sequences; or has at least 80% of sequence identity with the complementarity determining region of the following amino acid sequences and an affinity of K_{D≤}1.5647×10⁻⁹ mol/L with a pan-species-specific plasmodium lactate dehydrogenase;

a complementarity determining region CDR-VH1 is G-X1-S-F-T-N-Y-X2-M-N, herein

X1 is S, Y or T, and X2 is W or F;

a complementarity determining region CDR-VH2 is I-X1-P-S-X2-S-E-T-R-X3-N-Q, herein

X1 is H or N, X2 is E or D, and X3 is I, V or L;

a complementarity determining region CDR-VH3 is A-X1-S-G-X2-F-Y-T-X3-Y-X4-D-Y, herein,

X1 is K or R, X2 is D or E, X3 is S, Y or T, and X4 is F or W;

a complementarity determining region CDR-VL1 is R-G-X1-G-N-X2-H-N-Y-X3-A, herein,

X1 is S or T, X2 is 1, V or L, and X3 is I or L;

a complementarity determining region CDR-VL2 is N-A-X1-T-X2-A-D, herein

X1 is R or K, and X2 is I, V or L;

a complementarity determining region CDR-VL3 is Q-X1-F-W-S-X2-Y-T, herein

X1 is S, Y or T, and X2 is S or T.

The antibody may be used to qualitatively and quantitatively detect a pan-species-specific plasmodium lactate dehydrogenase (panLDH) in a sample, and is suitable for auxiliary diagnosis of a suspected malaria patient or screening and inspection of malaria cases.

The lactate dehydrogenases of Plasmodium falciparum, Plasmodium vivax, Plasmodium malariae or Plasmodium ovale have a high conservative property. The antibody provided in the present disclosure is pan-specific, and may bind to the lactate dehydrogenases of the above four types of the plasmodium.

In one or more implementation modes, the antigen binding domain has at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% of sequence identity with complementarity determining regions of the following amino acid sequences and has an affinity of K_{D}≤1.5647×10⁻⁹ mol/L, for example 1×10⁻⁹ mol/L, 2×10⁻⁹ mol/L, 3×10⁻⁹ mol/L, 4×10⁻⁹ mol/L, 4.5×10⁻⁹ mol/L, 5×10⁻⁹ mol/L, 6×10⁻⁹ mol/L, 7×10⁻⁹ mol/L, 8×10⁻⁹ mol/L, 9×10⁻⁹ mol/L, 1×10⁻¹⁰ mol/L, 3×10⁻¹⁰ mol/L, 5×10⁻¹⁰ mol/L, 7×10⁻¹⁰ mol/L, 9×10⁻¹⁰ mol/L or 1×10⁻¹¹ mol/L, 2×10⁻¹¹, 3×10⁻¹¹, 4×10⁻¹¹, 5×10⁻¹¹, 6×10⁻¹¹, 7×10⁻¹¹, 8×10⁻¹¹, 9×10⁻¹¹, or the K_{D} less than or equal to 1×10⁻⁹ mol/L, 2×10⁻⁹ mol/L, 3×10⁻⁹ mol/L, 4×10⁻⁹ mol/L, 4.5×10⁻⁹ mol/L, 5×10⁻⁹ mol/L, 6×10⁻⁹ mol/L, 7×10⁻⁹ mol/L, 8×10⁻⁹ mol/L, 9×10⁻⁹ mol/L, 1×10⁻¹⁰ mol/L, 3×10⁻¹⁰ mol/L, 5×10⁻¹⁰ mol/L, 7×10⁻¹⁰ mol/L, 9×10⁻¹⁰ mol/L or 1×10⁻¹¹ mol/L, 2×10⁻¹¹, 3×10⁻¹¹, 4×10⁻¹¹, 5×10⁻¹¹, 6×10⁻¹¹, 7×10⁻¹¹, 8×10⁻¹¹ or 9×10⁻¹¹;

or 8.7941×10⁻¹¹ mol/L K_{D}≤1.5647×10⁻⁹ mol/L with a pan-species-specific antigen plasmodium lactate dehydrogenase;

herein, the affinity is measured according to a method in the description of the present disclosure.

In one or more implementation modes:

in the complementarity determining region CDR-VH1, the X2 is W;

in the complementarity determining region CDR-VH2, the X1 is H;

in the complementarity determining region CDR-VH3, the X4 is F;

in the complementarity determining region CDR-VL1, the X3 is L; and

in the complementarity determining region CDR-VL3, the X2 is T.

In one or more implementation modes, in the complementarity determining region CDR-VH1, the X1 is S.

In one or more implementation modes, in the complementarity determining region CDR-VH1, the X1 is Y.

In one or more implementation modes, in the complementarity determining region CDR-VH1, the X1 is T.

In one or more implementation modes, in the complementarity determining region CDR-VH2, the X2 is E.

In one or more implementation modes, in the complementarity determining region CDR-VH2, the X2 is D.

In one or more implementation modes, in the complementarity determining region CDR-VH2, the X3 is I.

In one or more implementation modes, in the complementarity determining region CDR-VH2, the X3 is V.

In one or more implementation modes, in the complementarity determining region CDR-VH2, the X3 is L.

In one or more implementation modes, in the complementarity determining region CDR-VH3, the X1 is K.

In one or more implementation modes, in the complementarity determining region CDR-VH3, the X1 is R.

In one or more implementation modes, in the complementarity determining region CDR-VH3, the X2 is D.

In one or more implementation modes, in the complementarity determining region CDR-VH3, the X2 is E.

In one or more implementation modes, in the complementarity determining region CDR-VH3, the X3 is S.

In one or more implementation modes, in the complementarity determining region CDR-VH3, the X3 is Y.

In one or more implementation modes, in the complementarity determining region CDR-VH3, the X3 is T.

In one or more implementation modes, in the complementarity determining region CDR-VL1, the X1 is S.

In one or more implementation modes, in the complementarity determining region CDR-VL1, the X1 is T.

In one or more implementation modes, in the complementarity determining region CDR-VL1, the X2 is I.

In one or more implementation modes, in the complementarity determining region CDR-VL1, the X2 is V.

In one or more implementation modes, in the complementarity determining region CDR-VL1, the X2 is L.

In one or more implementation modes, in the complementarity determining region CDR-VL2, the X1 is R.

In one or more implementation modes, in the complementarity determining region CDR-VL2, the X1 is K.

In one or more implementation modes, in the complementarity determining region CDR-VL2, the X2 is I.

In one or more implementation modes, in the complementarity determining region CDR-VL2, the X2 is V.

In one or more implementation modes, in the complementarity determining region CDR-VL2, the X2 is L.

In one or more implementation modes, in the complementarity determining region CDR-VL3, the X1 is S.

In one or more implementation modes, in the complementarity determining region CDR-VL3, the X1 is Y.

In one or more implementation modes, in the complementarity determining region CDR-VL3, the X1 is T.

In one or more implementation modes, mutation sites of each complementarity determining region are selected from any one of the following mutation combinations:

| | CDR-VH1 | CDR-VH2 | CDR-VH3 | CDR-VL1 | CDR-VL2 | CDR-VL3 |
|---|---|---|---|---|---|---|
| Site | X1 | X2/X3 | X1/X2/X3 | X1/X2 | X1/X2 | X1 |
| Mutation | S | E/I | K/D/S | S/L | R/I | S |
| combination 1 | | | | | | |
| Mutation | Y | E/L | K/D/Y | T/L | R/V | Y |
| combination 2 | | | | | | |
| Mutation | T | E/V | K/D/T | S/V | R/L | T |
| combination 3 | | | | | | |
| Mutation | T | D/I | K/E/S | T/V | K/I | Y |
| combination 4 | | | | | | |
| Mutation | Y | D/L | K/E/Y | S/I | K/V | T |
| combination 5 | | | | | | |
| Mutation | S | D/V | K/E/T | T/I | K/L | S |
| combination 6 | | | | | | |
| Mutation | T | D/I | R/D/S | T/I | K/L | T |
| combination 7 | | | | | | |
| Mutation | S | D/L | R/D/Y | S/I | K/V | S |
| combination 8 | | | | | | |
| Mutation | Y | D/V | R/D/T | T/V | K/I | Y |
| combination 9 | | | | | | |
| Mutation | S | E/I | R/E/S | S/V | R/L | S |
| combination 10 | | | | | | |
| Mutation | T | E/L | R/E/Y | T/L | R/V | Y |
| combination 11 | | | | | | |
| Mutation | Y | E/V | R/E/T | S/L | R/I | T |
| combination 12 | | | | | | |
| Mutation | Y | D/I | K/D/S | S/L | K/V | Y |
| combination 13 | | | | | | |
| Mutation | T | D/L | K/D/Y | T/L | K/L | T |
| combination 14 | | | | | | |
| Mutation | S | D/V | K/D/T | S/V | K/I | S |
| combination 15 | | | | | | |
| Mutation | S | E/I | K/E/S | T/V | R/V | T |
| combination 16 | | | | | | |
| Mutation | Y | E/L | K/E/Y | S/I | R/L | S |
| combination 17 | | | | | | |
| Mutation | T | E/V | K/E/T | T/I | R/V | T |
| combination 18 | | | | | | |
| Mutation | T | D/I | R/D/S | T/I | K/I | S |
| combination 19 | | | | | | |
| Mutation | Y | D/L | R/D/Y | S/I | K/L | Y |
| combination 20 | | | | | | |
| Mutation | S | D/V | R/D/T | T/V | KV | T |
| combination 21 | | | | | | |
| Mutation | T | E/I | R/E/S | S/V | R/I | Y |
| combination 22 | | | | | | |
| Mutation | S | E/L | R/E/Y | T/L | R/L | T |
| combination 23 | | | | | | |
| Mutation | Y | E/V | R/E/T | S/L | R/V | S |
| combination 24 | | | | | | |
| Mutation | S | D/I | K/D/S | S/L | K/L | T |
| combination 25 | | | | | | |
| Mutation | T | D/L | K/D/Y | T/L | K/I | S |
| combination 26 | | | | | | |
| Mutation | Y | D/V | K/D/T | S/V | K/V | Y |
| combination 27 | | | | | | |
| Mutation | Y | E/I | K/E/S | T/V | R/L | S |
| combination 28 | | | | | | |
| Mutation | T | E/L | K/E/Y | S/I | R/I | Y |
| combination 29 | | | | | | |
| Mutation | S | E/V | K/E/T | T/I | R/V | T |
| combination 30 | | | | | | |
| Mutation | S | D/I | R/D/S | T/I | K/L | Y |
| combination 31 | | | | | | |
| Mutation | Y | D/L | R/D/Y | S/I | K/V | T |
| combination 32 | | | | | | |
| Mutation | T | D/V | R/D/T | T/V | K/I | S |
| combination 33 | | | | | | |
| Mutation | T | E/I | R/E/S | S/V | R/L | T |
| combination 34 | | | | | | |
| Mutation | Y | E/L | R/E/Y | T/L | R/V | S |
| combination 35 | | | | | | |
| Mutation | S | E/V | R/E/T | S/I | R/I | Y |
| combination 36 | | | | | | |
| Mutation | T | D/I | K/D/S | S/L | K/V | S |
| combination 37 | | | | | | |
| Mutation | S | D/L | K/D/Y | T/L | K/I | Y |
| combination 38 | | | | | | |
| Mutation | Y | D/V | K/D/T | S/V | K/L | T |
| combination 39 | | | | | | |
| Mutation | S | E/I | K/E/S | T/V | R/V | Y |
| combination 40 | | | | | | |
| Mutation | T | E/L | K/E/Y | S/I | R/I | T |
| combination 41 | | | | | | |
| Mutation | Y | E/V | K/E/T | T/I | R/L | S |
| combination 42 | | | | | | |
| Mutation | Y | D/I | R/D/S | T/I | K/I | T |
| combination 43 | | | | | | |
| Mutation | T | D/L | R/D/Y | S/I | K/V | S |
| combination 44 | | | | | | |
| Mutation | S | D/V | R/E/S | T/V | K/L | Y |
| combination 45 | | | | | | |
| Mutation | Y | E/I | R/E/Y | S/V | R/I | S |
| combination 46 | | | | | | |
| Mutation | T | E/L | R/E/T | T/L | R/V | Y |
| combination 47 | | | | | | |
| Mutation | T | E/V | K/D/S | S/L | R/L | T |
| combination 48 | | | | | | |
| Mutation | Y | D/I | K/D/Y | S/L | K/L | Y |
| combination 49 | | | | | | |
| Mutation | S | D/L | K/D/T | T/L | K/V | T |
| combination 50 | | | | | | |
| Mutation | T | D/V | K/E/S | S/V | K/I | S |
| combination 51 | | | | | | |
| Mutation | S | E/I | K/E/Y | T/V | R/L | T |
| combination 52 | | | | | | |
| Mutation | Y | E/L | K/E/T | S/I | R/V | S |
| combination 53 | | | | | | |
| Mutation | S | E/V | R/D/S | T/I | R/I | Y |
| combination 54 | | | | | | |
| Mutation | T | D/I | R/D/Y | T/I | K/V | S |
| combination 55 | | | | | | |
| Mutation | Y | D/L | R/D/T | S/I | K/I | Y |
| combination 56 | | | | | | |

In one or more implementation modes, the X1 which appears in the six CDR regions of the binding protein of the present disclosure each independently represents an amino acid defined in the present disclosure; the X2 which appears in the six CDR regions of the binding protein of the present disclosure each independently represents an amino acid defined in the present disclosure; the X3 which appears in the six CDR regions of the binding protein of the present disclosure each independently represents an amino acid defined in the present disclosure; and the X4 which appears in the six CDR regions of the binding protein of the present disclosure each independently represents an amino acid defined in the present disclosure.

In one or more implementation modes, the binding protein includes at least 3 GDRs (for example, 3 light chain CDRs or 3 heavy chain CDRs); or, the binding protein includes at least 6 CDRs.

In one or more implementation modes, the binding protein is a complete antibody containing a variable region and a constant region.

In one or more implementation modes, the binding protein is a “functional fragment” of the antibody, for example, one of a nano-antibody, F(ab′)₂, Fab′, Fab, Fv, scFv, diabodies, and an antibody minimum recognition unit.

scFv (sc=single chain), diabodies.

The “functional fragment” described in the present disclosure particularly refers to an antibody fragment having the same specificity as a maternal antibody for the pan-species-specific antigen plasmodium lactate dehydrogenase. In addition to the above functional fragments, any fragments of which a half-life is increased are also included.

These functional fragments usually have the same binding specificity as the antibody from which they are derived. Those skilled in the art infer from the content recorded in the present disclosure that the antibody fragments of the present disclosure may obtain the above functional fragments by methods such as enzymatic digestion (including pepsin or papain) and/or by methods of chemical reduction and splitting of disulfide bonds.

The antibody fragment may also be obtained by peptide synthesis through a recombinant genetic technology that is also known to those skilled in the art or through, for example, an automated peptide synthesizer, such as automated peptide synthesizers sold by Applied BioSystems.

In one or more implementation modes, the binding protein includes sequences of light chain framework regions FR-L1, FR-L2, FR-L3 and FR-L4 successively shown in SEQ ID NO: 1-4, and/or sequences of heavy chain framework regions FR-H1, FR-H2, FR-H3 and FR-H4 successively shown in SEQ ID NO: 5-8.

In one or more implementation modes, the binding protein further includes an antibody constant region sequence.

In one or more implementation modes, the constant region sequence is selected from a sequence of a constant region of any one of IgG1, IgG2, IgG3, IgG4, IgA, IgM, IgE, and IgD;

In one or more implementation modes, the species source of the constant region is cattle, horse, dairy cow, pig, sheep, goat, rat, mouse, dog, cat, rabbit, camel, donkey, deer, mink, chicken, duck, goose, turkey, gamecock or human.

In one or more implementation modes, the constant region is derived from the mouse;

a light chain constant region sequence is shown in SEQ ID NO: 9; and

a heavy chain constant region sequence is shown in SEQ ID NO: 10.

According to one aspect of the present disclosure, the present disclosure further relates to an isolated nucleic acid molecule, the nucleic acid molecule is DNA or RNA, and it encodes the binding protein as mentioned above.

According to one aspect of the present disclosure, the present disclosure further relates to a vector, and it includes the nucleic acid molecule as mentioned above.

The present disclosure further includes at least one nucleic construct encoding the nucleic acid molecule as mentioned above, such as a plasmid, and further an expression plasmid, A construction method for the vector may be introduced in an embodiment of the present application.

According to one aspect of the present disclosure, the present disclosure further relates to a host cell, and it is transformed by the vector as mentioned above.

The host cell may be a eukaryotic cell, such as a mammalian cell.

In one or more implementation modes, the host cell is a CHO cell.

According to one aspect of the present disclosure, the present disclosure further relates to a method for producing the binding protein as mentioned above, the method includes the following steps:

culturing the host cell as mentioned above in a culture medium and under a suitable culture condition, and recovering the binding protein thus produced from the culture medium or from the cultured host cell.

According to one aspect of the present disclosure, the present disclosure further relates to an application of the binding protein as mentioned above in preparing a diagnostic agent for diagnosing malaria.

According to one aspect of the present disclosure, the present disclosure further relates to a method for detecting a pan-species-specific antigen plasmodium lactate dehydrogenase in a test sample, and it includes:

a) under a condition sufficient for an antibody/antigen binding reaction to occur, contacting a pan-species-specific antigen plasmodium lactate dehydrogenase antigen with the binding protein as mentioned above so as to form an immune complex; and

b) detecting the presence of the immune complex, the presence of the complex indicates the presence of the pan-species-specific antigen plasmodium lactate dehydrogenase in the test sample.

In this implementation mode, the binding protein may be labeled with an indicator that shows signal intensity, so that the complex may be easily detected.

In one or more implementation modes, in the step a), the immune complex further includes a second antibody, and the second antibody binds to the binding protein.

In one or more implementation modes, in the step a), the immune complex further includes a second antibody, and the second antibody binds to the pan-species-specific antigen plasmodium lactate dehydrogenase.

In this implementation mode, the binding protein in the form of a first antibody forms a paired antibody with the second antibody, and is used for binding to different epitopes of the pan-species-specific antigen plasmodium lactate dehydrogenase; and

the second antibody may be labeled with an indicator that shows signal intensity, so that the complex may be easily detected.

In one or more implementation modes, in the step a), the immune complex further includes a second antibody, the second antibody binds to the pan-species-specific antigen plasmodium lactate dehydrogenase antigen; and

in this implementation mode, the binding protein is served as the antigen of the second antibody, and the second antibody may be labeled with an indicator that shows signal intensity, so that the complex may be easily detected.

In one or more implementation modes, the indicator for showing the signal intensity includes any one of a fluorescent substance, a quantum dot, a digoxigenin-labeled probe, a biotin, a radioisotope, a radiocontrast agent, a paramagnetic ion fluorescent microsphere, an electron dense substance, a chemiluminescent marker, an ultrasound contrast agent, a photosensitizer, a colloidal gold or an enzyme.

In one or more implementation modes, the fluorescent substance includes any one of Alexa 350, Alexa 405, Alexa 430, Alexa 488, Alexa 555, Alexa 647, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxyfluorescein, 5-carboxy-2′,4′,5′,7′-tetrachlorofluorescein, 5-carboxyfluorescein, 5-carboxyrhodamine, 6-carboxyrhodamine, 6-carboxytetramethylrhodamine, Cascade Blue, Cy2, Cy3, Cy5, Cy7, 6-FAM, dansyl chloride, fluorescein, HEX, 6-JOE, NBD (7-nitrobenzo-2-oxa-1,3-diazole), Oregon Green 488, Oregon Green 500, Oregon Green 514, Pacific Blue, phthalic acid, terephthalic acid, isophthalic acid, cresol solid violet, cresol blue violet, brilliant cresol blue, p-aminobenzoic acid, erythrosine, phthalocyanine, azomethine, cyanine, xanthine, succinylfluorescein, rare earth metal cryptate, tris-bispyridyldiamine europium, europium cryptate or chelate, diamine, biscyanin, La Jolla blue dye, allophycocyanin, allococyanin B, phycocyanin C, phycocyanin R, thiamine, phycoerythrin, phycoerythrin R, REG, rhodamine green, rhodamine isothiocyanate, rhodamine red, ROX, TAMRA, TET, TRIT (tetramethylrhodamine isothiol), tetramethylrhodamine and Texas Red.

In one or more implementation modes, the radioisotope includes any one of ¹¹⁰In, ¹¹¹In, ¹⁷⁷Lu, ¹⁸F, ²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ⁹⁴mTc, ⁹⁴Tc, ⁹⁹mTc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb and ⁸³Sr.

In one or more implementation modes, the enzyme includes any one of horseradish peroxidase, alkaline phosphatase, and glucose oxidase.

In one or more implementation modes, the fluorescent microsphere is: a polystyrene fluorescent microsphere, and the interior is wrapped with the rare earth fluorescent ion europium.

According to one aspect of the present disclosure, the present disclosure further relates to a kit, and it includes the binding protein as mentioned above.

The present disclosure further provides an application of the binding protein described herein in diagnosis of malaria.

The present disclosure further provides a method for diagnosing malaria, including:

A) under a condition sufficient for a binding reaction to occur, contacting a sample from a subject with the binding protein of the present disclosure so as to perform the binding reaction; and

B) detecting an immune complex produced by the binding reaction,

herein the presence of the immune complex indicates the presence of the malaria.

In one or more implementation modes, the method is based on a fluorescence immunoassay technology, a chemiluminescence technology, a colloidal gold immunoassay technology, a radioimmunoassay and/or an enzyme-linked immunoassay technology.

In one or more implementation modes, the sample is selected from at least one of whole blood, peripheral blood, serum or plasma.

In one or more implementation modes, the subject is a mammal, preferably a primate, more preferably a human.

In one or more implementation modes, the malaria is selected from a group consisting of Plasmodium vivax, Plasmodium falciparum, Plasmodium malariae, Plasmodium ovale or a combination thereof.

In one or more implementation modes, the malaria is a malaria caused by Plasmodium.

The implementation schemes of the present disclosure are described in detail below in combination with embodiments, but those skilled in the art may understand that the following embodiments are only used to illustrate the present disclosure, and should not be regarded as limitation to a scope of the present disclosure. If specific conditions are not indicated in the embodiments, it shall be performed in accordance with conventional conditions or conditions recommended by a manufacturer. Used reagents or instruments in which the manufacturer is not indicated are all conventional products that may be purchased commercially.

### Embodiment 1

This embodiment provides an exemplary preparation method for a recombinant antibody against a pan-species-specific antigen plasmodium lactate dehydrogenase.

S1. Construction of Expression Plasmid:

In this embodiment, restriction endonuclease and Prime Star DNA polymerase are purchased from Takara Company;

Mag Extractor-RNA extraction kit is purchased from TOYOBO Company;

BD SMART™ RACE cDNA Amplification Kit is purchased from Takara Company;

pMD-18T vector is purchased from Takara Company;

plasmid extraction kit is purchased from Tiangen Company;

primer synthesis and gene sequencing are completed by Invitrogen Company; and

secreting Anti-PAN-PLDH monoclonal antibody is an existing hybridoma cell line, and it is resuscitated for later use.

S11. Design and Synthesis of Primers:

5′RACE upstream primers for amplifying heavy chain and light chain: SMARTER II A oligonucleotide:

5′-AAGCAGTGGTATCAACGCAGAGTACXXXXX-3′;

5′-RACE CDS primer (5′-CDS): 5′-(T)₂₅VN-3′(N=A, C, G, or T; V=A, G, or C);

Universal Primer A Mixture (UPM):

5′-CTAATACGACTCACTATAGGGCAAGCAGTGGTATCAACGCAGAGT-
3′;

Nested universal primer A (NUP):

5′-AAGCAGTGGTATCAACGCAGAGT-3′;
mkR:
5′-CGCCTAACACTCATTCCTGTTGAAGC-3′;
mHR:
5′-CCGCTCATTTACCCGGAGACCG-3′.

S12. Antibody Variable Region Gene Cloning and Sequencing:

RNA is extracted from the hybridoma cell line secreting the anti-Pan-PLDH 9G7 monoclonal antibody, and first chain cDNA synthesis is performed with SMARTER™ RACE cDNA Amplification Kit and SMARTER II A oligonucleotide and 5′-CDS primers in the kit, and an obtained first chain cDNA product is used as a PCR amplification template. Light chain genes are amplified with the universal primer A mixture (UPM), the nested universal primer A (NUP) and the mkR primer, and heavy chain genes are amplified with the universal primer A mixture (UPM), the nested universal primer A (NUP) and the mHR primer. Herein about 0.7 KB of a target band is amplified by a primer pair of the light chain, and about 1.5 KB of a target band is amplified by a primer pair of the heavy chain. Purification and recovery are performed by agarose gel electrophoresis, a product is subjected to an A-adding reaction with rTaq DNA polymerase and inserted into the pMD-18T vector, and transformed into a DH5a competent cell. After bacterial colonies are grew, 10 of the heavy chain and light chain genes clones each are taken separately and sent to Invitrogen Company for sequencing.

S13. Sequence Analysis of Variable Region Genes of Anti-PAN-PLDH 9G7 Antibody:

The gene sequence obtained by the above sequencing is put in the IMGT antibody database for analysis, and VNT111.5 software is used for analyzing to determine that the genes amplified by the heavy chain and light chain primers are all correct, herein in the gene fragment amplified by the light chain, the VL gene sequence is 375 bp, and belongs to a VkII gene family, there is 57 bp of a leader peptide sequence in front of it; and in the gene fragment amplified by the heavy chain primer pair, the VH gene sequence is 417 bp, and belongs to a VH1 gene family, there is 57 bp of a leader peptide sequence in front of it.

S14. Construction of Recombinant Antibody Expression Plasmid:

The pcDNA™ 3.4 TOPO® vector is a constructed recombinant antibody eukaryotic expression vector. The polyclonal restriction sites such as HindIII, BamHI, and EcoRI have already been introduced into the expression vector, and is named as pcDNA3.4A expression vector, and then referred to as 3.4A expression vector; Based on the above sequencing results of antibody variable region genes in pMD-18T, specific primers for VL and VH genes of anti-Pan-PLDH 9G7 are designed, HindIII and EcoRI restriction sites and protective bases are respectively provided at both ends, and the primers are as follows:

Pan-9G7-HF:
5′-CCCAAGCTTGCCGCCACCATGAGTGTGCTCACTCAGGTCCTGGGGT-
3′;
Pan-9G7-HR:
5′-GGGGAATTCTCATTTACCCGGAGACCGGGAGATGGTCTTC-3′;
Pan-9G7-LF:
5′-CCCAAGCTTGCCGCCACCATGAAGTCACAGACCCAGGTCTTCGTA-
3′;
Pan-9G7-LR:
5′-CCCGAATTCTCAACACTCATTCCTGTTGAAGCTCTTGACGATG-3′;

723 bp of a light chain gene fragment and 1.452 kb of a heavy chain gene fragment are amplified by a PCR amplification method. The heavy chain and light chain gene fragments are double-digested with HindIII/EcoRI, and the 3.4A vector is double-digested with HindIII/EcoRI. After the fragments and vector are purified and recovered, the heavy chain gene and the light chain gene are linked to the 3.4A expression vector respectively, recombinant expression plasmids of the heavy chain and the light chain are obtained respectively.

S2. Antibody Preparation

S21. Transient Transfection of Recombinant Plasmid into CHO Cells

The plasmid is diluted with ultrapure water to 400 μg/ml, CHO cells are adjusted to 1.7×10⁷ cells/ml in a centrifuge tube, 100 μl of the plasmid is mixed with 700 μl of the cells, and transferred to an electroporation cup, electroporated, and transferred to 10 ml of CD CHO AGT-containing medium. It is cultured in a shaker at 37° C. (8% CO₂, 115-200 rpm of vibration amplitude); a sample is taken every day to detect cell viability. While the cell viability is lower than 50%, the cells are centrifuged and supernatant is cultured.

S22. Antibody Activity Identification of Expression Supernatant

A Pan-PLDH protein is diluted with coating solution to a specified concentration, 100 uL per well, and overnight at 4° C.; on the next day, it is washed twice with a washing solution, and patted dry; blocking solution (20% BSA+80% PBS) is added, 120 uL per well, and incubated at 37° C. for 1 h and patted dry; the cell supernatant after doubling dilution is added, 100 uL/well, and incubated at 37° C. for 30 min (partial supernatant 1 h); it is washed with the washing solution for 5 times and patted dry; goat anti-mouse IgG-HRP is added, 100 uL/well, 37° C., and 30 min; it is washed with the washing solution for 5 times, and patted dry; color developing solution A (50 uL/well) is added, and color developing solution B (50 uL/well) is added, 10 min; stop solution is added, 50 uL/well; and an OD value is read at 450 nm (refer to 630 nm) on a microplate reader. After identification, the antibodies produced after transient transfection of the constructed expression plasmid are all active to the Pan-PLDH protein.

Purification of Expression Supernatant by Protein A Affinity Chromatography Column

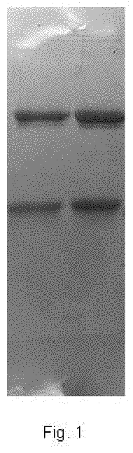

Supernatant of fermentation solution is taken and filtered with a 0.22 μm membrane. The supernatant passes through a Mab Slelect SuRe LX (GE Healthcare) affinity packing column at a certain flow rate and is hanged on the column, and then 20 mM NaAc (pH3.4) solution is used for elution, and a certain amount of 1 M Tris solution is added to a sample collection tube for pre-neutralization. The eluted sample is dialyzed and solution-changed in PBS (pH 7.4) solution for three times and then the purified antibody is obtained. The purified antibody is taken for reducibility SDS-PAGE, Results are shown in FIG. 1, the first lane is 0.5 mg/ml and the second lane is 1 mg/ml, herein the chain with a larger molecular weight is the heavy chain, and the chain with a smaller molecular weight is the light chain.

### Embodiment 2

Antibody affinity analysis and activity identification
The antibody obtained in Embodiment t has sequences of a light chain as shown in SEQ ID NO: 11 and a heavy chain as shown in 12 after being analyzed.

Sequences of SEQ ID NO:1-12 are as follows:

| | | SEQ |
|---|---|---|
| Sequence name | Sequence | ID NO |
| Light chain framework region | DIQLTQSPASLSASVGETVTITC | 1 |
| FR-L1 | | |
| Light chain framework region | WYQQKQGKSPQLLVY | 2 |
| FR-L2 | | |
| Light chain framework region | GVPSRFSGSGSGTQYSLKINSLQPEDFGSYYC | 3 |
| FR-L3 | | |
| Light chain framework region | FGGGTKLEIK | 4 |
| FR-L4 | | |
| Heavy chain framework region | QVQLQQPGAELVRPGASVKLSCKAS | 5 |
| FR-H1 | | |
| Heavy chain framework region | WVKQRPGQGLEWIGM | 6 |
| FR-H2 | | |
| Heavy chain framework region | KFKDKATLTVDKSSSTAYMQLSSLTAEDSAVYYC | 7 |
| FR-H3 | | |
| Heavy chain framework region | WGQGTTLTVSS | 8 |
| FR-H4 | | |
| Light chain constant region | RADAAPTVSIFPPSSEQLTSGGASVVCFLNNFYPK | 9 |
| | DINVKWKIDGSERQNGVLNSWTDQDSKDSTYSM | |
| | SSTLTLTKDEYERHNSYTCEATHKTSTSPIVKSFNR | |
| | NEC | |
| Heavy chain constant region | AKTTPPSVYPLAPGCGDTTGSSVTLGCLVKGYFP | 10 |
| | ESVTVTWNSGSLSSSVHTFPALLQSGLYTMSSSV | |
| | TVPSSTWPSQTVTCSVAHPASSTTVDKKLEPSGPI | |
| | STINPCPPCKECHKCPAPNLEGGPSVFIFPPNIKDV | |
| | LMISLTPKVTCVVVDVSEDDPDVQISWFVNNVEVH | |
| | TAQTQTHREDYNSTIRVVSTLPIQHQDWMSGKEF | |
| | KCKVNNKDLPSPIERTISKIKGLVRAPQVYILPPPAE | |
| | QLSRKDVSLTCLVVGFNPGDISVEWTSNGHTEEN | |
| | YKDTAPVLDSDGSYFIYSKLNMKTSKWEKTDSFS | |
| | CNVRHEGLKNYYLKKTISRSPG | |
| Light chain | DIQLTQSPASLSASVGETVTITCRGSGNLHNYIAW | 11 |
| | YQQKQGKSPQLLVYNARTIADGVPSRFSGSGSGT | |
| | QYSLKINSLQPEDFGSYYCQSFWSSYTFGGGTKL | |
| | EIKRADAAPTVSIFPPSSEQLTSGGASVVCFLNNF | |
| | YPKDINVKWKIDGSERQNGVLNSWTDQDSKDSTY | |
| | SMSSTLTLTKDEYERHNSYTCEATHKTSTSPIVKS | |
| | FNRNEC | |
| Heavy chain | QVQLQQPGAELVRPGASVKLSCKASGSSFTNYFM | 12 |
| | NWVKQRPGQGLEWIGMINPSESETRINQKFKDKA | |
| | TLTVDKSSSTAYMQLSSLTAEDSAVYYCAKSGDFY | |
| | TSYWDYWGQGTTLTVSSAKTTPPSVYPLAPGCG | |
| | DTTGSSVTLGCLVKGYFPESVTVTWNSGSLSSSV | |
| | HTFPALLQSGLYTMSSSVTVPSSTWPSQTVTCSV | |
| | AHPASSTTVDKKLEPSGPISTINPCPPCKECHKCP | |
| | APNLEGGPSVFIFPPNIKDVLMISLTPKVTCVVVDV | |
| | SEDDPDVQISWFVNNVEVHTAQTQTHREDYNSTI | |
| | RVVSTLPIQHQDEMSGKEFKCKVNNKDLPSPIER | |
| | TISKIKGLVRAPQVYILPPPAEQLSRKDVSLTCLVVG | |
| | FNPGDISVEWTSNGHTEENYKDTAPVLDSDGSYFI | |
| | YSKLNMKTSKWEKTDSFSCNVRHEGLKNYYLKKT | |
| | ISRSPG | |

After analysis, complementarity determining regions (WT) of the heavy chain:

CDR-VH1 is G-S(X1)-S-F-T-N-Y-F(X2)-M-N;

CDR-VH2 is I-N(X1)-P-S-E(X2)-S-E-T-R-I(X3)-N-Q;

CDR-VH3 is A-K(X1)-S-G-D(X2)-F-Y-T-S(X3)-Y-W(X4)-D-Y;

complementarity determining regions of the light chain:

CDR-VL1 is R-G-S(X1)-G-N-L(X2)-H-N-Y-I(X3)-A;

CDR-VL2 is N-A-R(X1)-T-I(X2)-A-D;

CDR-VL3 is Q-S(X1)-F-W-S-S(X2)-Y-T;

herein, the X1, X2, and X3 are all sites to be mutated.

TABLE 1
Mutation sites related to antibody activity

| | CDR-VH1 | CDR-VH2 | CDR-VH3 | CDR-VL1 | CDR-VL3 |
|---|---|---|---|---|---|
| Site | X2 | X1 | X4 | X3 | X2 |
| WT | F | N | W | I | S |
| Mutation 1 | W | H | F | L | T |
| Mutation 2 | F | H | F | L | S |
| Mutation 3 | W | N | F | I | T |
| Mutation 4 | F | H | W | I | T |

The above mutations are performed on CDR sites in WT by the inventor, to obtain the antibody with the better activity.

A recombinant MA protein (self-produced 150520-1) is diluted with coating solution to 1 μg/ml for microplate coating, 100 uL per well, and overnight at 4° C.; on the next day, it is washed with the washing solution twice, and patted dry; blocking solution (20% BSA+80% PBS) is added, 120 uL per well, 37° C., and 1 h, and patted dry; the diluted MA monoclonal antibody is added, 100 uL/well, 37° C., and 30 min (partial supernatant 1 h); it is washed with the washing solution for 5 times, and patted dry; goat anti-mouse IgG-HRP is added, 100 uL per well, 37° C., and 30 min; it is washed with the washing solution for 5 times, and patted dry; color developing solution A (50 uL/well) is added, and color developing solution B (50 uL/well) is added, 10 min; stop solution is added, 50 uL/well; an OD value is read at 450 nm (refer to 630 nm) on a microplate reader.

TABLE 2
Antibody activity analysis data

| Sample | | | | | |
|---|---|---|---|---|---|
| concentration | | Muta- | Muta- | Muta- | Muta- |
| ng/ml | WT | tion 1 | tion 2 | tion 3 | tion 4 |
| 1000 | 1.936 | 2.264 | 2.213 | 2.229 | 2.148 |
| 200 | 1.819 | 2.188 | 2.137 | 2.140 | 2.030 |
| 40 | 1.428 | 2.024 | 1.978 | 2.010 | 1.921 |
| 8 | 0.635 | 1.100 | 1.001 | 1.013 | 0.939 |
| 1.6 | 0.205 | 0.339 | 0.297 | 0.274 | 0.251 |
| 0.32 | 0.173 | 0.270 | 0.231 | 0.255 | 0.211 |
| 0 | 0.072 | 0.060 | 0.072 | 0.053 | 0.066 |

It may be seen from the above table that Mutation 1 has the best activity effect, so Mutation 1 is used as the framework sequence to screen for mutation sites with the better titer (it is guaranteed that the antibody activity obtained by screening is similar to that of Mutation 1, and the antibody activity is ±10%), some results are as follows.

TABLE 3
Mutation sites related to antibody affinity

| | CDR-VH1 | CDR-VH2 | CDR-VH3 | CDR-VL1 | CDR-VL2 | CDR-VL3 |
|---|---|---|---|---|---|---|
| Site | X1 | X2/X3 | X1/X2/X3 | X1/X2 | X1/X2 | X1 |
| Mutation 1 | S | E/I | K/D/S | S/L | R/I | S |
| Mutation 1-1 | Y | E/L | K/D/Y | T/L | R/V | Y |
| Mutation 1-2 | T | E/V | K/D/T | S/V | R/L | T |
| Mutation 1-3 | T | D/I | K/E/S | T/V | K/I | Y |
| Mutation 1-4 | Y | D/L | K/E/Y | S/I | K/V | T |
| Mutation 1-5 | S | D/V | K/E/T | T/I | K/L | S |
| Mutation 1-6 | T | D/I | R/D/S | T/I | K/L | T |
| Mutation 1-7 | S | D/L | R/D/Y | S/I | K/V | S |
| Mutation 1-8 | Y | D/V | R/D/T | T/V | K/I | Y |
| Mutation 1-9 | S | E/I | R/E/S | S/V | R/L | S |
| Mutation 1-10 | T | E/L | R/E/Y | T/L | R/V | Y |
| Mutation 1-11 | Y | E/V | R/E/T | S/L | R/I | T |
| Mutation 1-12 | Y | D/I | K/D/S | S/L | K/V | Y |
| Mutation 1-13 | T | D/L | K/D/Y | T/L | K/L | T |
| Mutation 1-14 | S | D/V | K/D/T | S/V | K/I | S |
| Mutation 1-15 | S | E/I | K/E/S | T/V | R/V | T |
| Mutation 1-16 | Y | E/L | K/E/Y | S/I | R/L | S |
| Mutation 1-17 | T | E/V | K/E/T | T/I | R/V | T |
| Mutation 1-18 | T | D/I | R/D/S | T/I | K/I | S |
| Mutation 1-19 | Y | D/L | R/D/Y | S/I | K/L | Y |
| Mutation 1-20 | S | D/V | R/D/T | T/V | KV | T |
| Mutation 1-21 | T | E/I | R/E/S | S/V | R/I | Y |
| Mutation 1-22 | S | E/L | R/E/Y | T/L | R/L | T |
| Mutation 1-23 | Y | E/V | R/E/T | S/L | R/V | S |
| Mutation 1-24 | S | D/I | K/D/S | S/L | K/L | T |
| Mutation 1-25 | T | D/L | K/D/Y | T/L | K/I | S |
| Mutation 1-26 | Y | D/V | K/D/T | S/V | K/V | Y |
| Mutation 1-27 | Y | E/I | K/E/S | T/V | R/L | S |
| Mutation 1-28 | T | E/L | K/E/Y | S/I | R/I | Y |
| Mutation 1-29 | S | E/V | K/E/T | T/I | R/V | T |
| Mutation 1-30 | S | D/I | R/D/S | T/I | K/L | Y |
| Mutation 1-31 | Y | D/L | R/D/Y | S/I | K/V | T |
| Mutation 1-32 | T | D/V | R/D/T | T/V | K/I | S |
| Mutation 1-33 | T | E/I | R/E/S | S/V | R/L | T |
| Mutation 1-34 | Y | E/L | R/E/Y | T/L | R/V | S |
| Mutation 1-35 | S | E/V | R/E/T | S/L | R/I | Y |
| Mutation 1-36 | T | D/I | K/D/S | S/L | K/V | S |
| Mutation 1-37 | S | D/L | K/D/Y | T/L | K/I | Y |
| Mutation 1-38 | Y | D/V | K/D/T | S/V | K/L | T |
| Mutation 1-39 | S | E/I | K/E/S | T/V | R/V | Y |
| Mutation 1-40 | T | E/L | K/E/Y | S/I | R/I | T |
| Mutation 1-41 | Y | E/V | K/E/T | T/I | R/L | S |
| Mutation 1-42 | Y | D/I | R/D/S | T/I | K/I | T |
| Mutation 1-43 | T | D/L | R/D/Y | S/I | K/V | S |
| Mutation 1-44 | S | D/V | R/E/S | T/V | K/L | Y |
| Mutation 1-45 | Y | E/I | R/E/Y | S/V | R/I | S |
| Mutation 1-46 | T | E/L | R/E/T | T/L | R/V | Y |
| Mutation 1-47 | T | E/V | K/D/S | S/L | R/L | T |
| Mutation 1-48 | Y | D/I | K/D/Y | S/L | K/L | Y |
| Mutation 1-49 | S | D/L | K/D/T | T/L | K/V | T |
| Mutation 1-50 | T | D/V | K/E/S | S/V | K/I | S |
| Mutation 1-51 | S | E/I | K/E/Y | T/V | R/L | T |
| Mutation 1-52 | Y | E/L | K/E/T | S/I | R/V | S |
| Mutation 1-53 | S | E/V | R/D/S | T/I | R/I | Y |
| Mutation 1-54 | T | D/I | R/D/Y | T/I | K/V | S |
| Mutation 1-55 | Y | D/L | R/D/T | S/I | K/I | Y |

Affinity Analysis

Data is made by an enzyme immunoassay indirect method in the same way as the activity identification, and coating is made into four gradients of 0.5 μg/ml, 0.25 μg/ml, 0.125 μg/ml, and 0.0625 μg/ml; the antibody is diluted by 2 times of the gradient from 100 ng/ml to 0.195 ng/ml and a sample is loaded. The 00 values corresponding to different antibody concentrations with different coating concentrations are obtained. Under the same coating concentration, the antibody concentrations are used as an abscissa, the 00 values are used as an ordinate, and a logarithmic plot is made. According to a filling equation, the antibody concentration at 50% of the maximum 00 value is calculated; it is assigned into a formula: K=(n−1)/(2×(n×Ab′−Ab)), a reciprocal of an affinity constant is calculated, herein Ab and Ab′ respectively represent the antibody concentrations at 50% of the maximum OD value in the corresponding coating concentrations (Ag, Ag′), n=Ag/Ag′; every two coating concentrations may be combined to calculate a K value, and finally six K values may be obtained. An average value thereof is taken and a reciprocal thereof is calculated to get the affinity constant K_{D}.

TABLE 4
Affinity analysis data

| | K_{D} |
|---|---|
| Mutation 1 | 4.1173E−10 |
| Mutation 1-1 | 5.207E−10 |
| Mutation 1-2 | 3.378E−10 |
| Mutation 1-3 | 9.8033E−11 |
| Mutation 1-4 | 5.9577E−10 |
| Mutation 1-5 | 5.325E−10 |
| Mutation 1-6 | 6.6414E−10 |
| Mutation 1-7 | 2.079E−10 |
| Mutation 1-8 | 4.8103E−10 |
| Mutation 1-9 | 6.1629E−10 |
| Mutation 1-10 | 5.2522E−10 |
| Mutation 1-11 | 4.6525E−10 |
| Mutation 1-12 | 1.1780E−10 |
| Mutation 1-13 | 5.3124E−10 |
| Mutation 1-14 | 4.8778E−10 |
| Mutation 1-15 | 4.6962E−10 |
| Mutation 1-16 | 4.1056E−10 |
| Mutation 1-17 | 3.7036E−10 |
| Mutation 1-18 | 5.6654E−10 |
| Mutation 1-19 | 3.2891E−10 |
| Mutation 1-20 | 2.0374E−10 |
| Mutation 1-21 | 6.5511E−10 |
| Mutation 1-22 | 3.7768E−10 |
| Mutation 1-23 | 3.1937E−10 |
| Mutation 1-24 | 4.0181E−10 |
| Mutation 1-25 | 4.9287E−10 |
| Mutation 1-26 | 3.7374E−10 |
| Mutation 1-27 | 4.2673E−10 |
| Mutation 1-28 | 7.5891E−10 |
| Mutation 1-29 | 4.1109E−10 |
| Mutation 1-30 | 1.9802E−10 |
| Mutation 1-31 | 5.1366E−10 |
| Mutation 1-32 | 3.2867E−10 |
| Mutation 1-33 | 3.7186E−10 |
| Mutation 1-34 | 5.6535E−10 |
| Mutation 1-35 | 4.2376E−10 |
| Mutation 1-36 | 5.1794E−10 |
| Mutation 1-37 | 5.1988E−10 |
| Mutation 1-38 | 6.1272E−10 |
| Mutation 1-39 | 5.4978E−10 |
| Mutation 1-40 | 4.3886E−10 |
| Mutation 1-41 | 7.4323E−10 |
| Mutation 1-42 | 5.4479E−10 |
| Mutation 1-43 | 5.2955E−10 |
| Mutation 1-44 | 3.1776E−10 |
| Mutation 1-45 | 4.1529E−10 |
| Mutation 1-46 | 3.3166E−10 |
| Mutation 1-47 | 4.5364E−10 |
| Mutation 1-48 | 3.362E−10 |
| Mutation 1-49 | 3.4246E−10 |
| Mutation 1-50 | 8.7941E−11 |
| Mutation 1-51 | 5.2612E−10 |
| Mutation 1-52 | 6.5918E−10 |
| Mutation 1-53 | 7.174E−10 |
| Mutation 1-54 | 6.326E−10 |
| Mutation 1-55 | 5.5916E−10 |

It may be seen from Table 4 that the mutation sites listed in Table 3 have little effect on the affinity of the antibody.

In order to verify the above result, the above experiment is repeated using WT as the framework sequence to verify the affinity of the mutation sites. Some results are as follows.

TABLE 5
Mutation using WT as framework

| | CDR-VH1 | CDR-VH2 | CDR-VH3 | CDR-VL1 | CDR-VL2 | CDR-VL3 |
|---|---|---|---|---|---|---|
| Site | X1 | X2/X3 | X1/X2/X3 | X1/X2 | X1/X2 | X1 |
| WT | S | E/I | K/D/S | S/L | R/I | S |
| WT 1-1 | T | D/I | R/D/S | T/I | K/L | T |
| WT 1-2 | Y | D/V | R/D/T | T/V | K/I | Y |
| WT 1-3 | S | E/I | R/E/Y | T/L | R/L | T |
| WT 1-4 | Y | D/V | K/D/T | S/V | K/V | Y |
| WT 1-5 | Y | E/V | K/E/T | T/I | R/L | S |
| WT 1-6 | T | D/L | R/D/Y | S/I | K/V | S |
| WT 1-7 | S | D/V | R/E/S | T/V | K/L | Y |
| WT 1-8 | T | D/V | K/E/S | S/V | K/I | S |
| WT 1-9 | S | E/I | K/E/Y | T/V | R/L | T |

TABLE 6
Affinity analysis data

| | K_{D} |
|---|---|
| WT | 8.8652E−10 |
| WT 1-1 | 7.5867E−10 |
| WT 1-2 | 6.3858E−10 |
| WT 1-3 | 4.8503E−10 |
| WT 1-4 | 8.4959E−10 |
| WT 1-5 | 5.1628E−10 |
| WT 1-6 | 1.5647E−09 |
| WT 1-7 | 7.5695E−10 |
| WT 1-8 | 1.3634E−09 |
| WT 1-9 | 6.2091E−10 |

It is analyzed from Table 5 and Table 6 that, under a precondition of guaranteeing the antibody activity, the above mutation sites have little correlation with other sites.

The above antibody in Table 4 and another strain of the internal antibody (antibody paired with the Pan-PLDH antibody) are subjected to a paired antibody experiment by the applicant to verify that the nature of the antibody is not changed significantly with the WT antibody, and it is verified by a double antibody sandwich method paired experiment that the specificities are all maintained at an original high level without any significant changes, it is indicated that the above antibody has the same epitope as that recognized by the WT antibody before mutation. Because the affinity of Mutation 1 is higher than that of the WT, a detection rate of Mutation 1 corresponding to the application of the kit is also higher than that of the WT. Furthermore, the specificity of the above antibody tested in an immunodiagnostic platform may reach 98%-100%, and the consistency of testing 100 samples may reach 95%-98%.

Furthermore, WT, Mutation 1, and 8 randomly selected mutant antibodies are tested for stability; the above antibodies are stored at 37° C. for 72 hours, and after being taken out, the same negative and positive quality control samples are detected under the same detection condition with the same batch of antibodies stored at 4° C. for 72 hours, and a detection method is the same as the antibody activity analysis method used in the above embodiment. The linearity of each group of the antibodies may reach 99.90% or more, and a CV value is less than 8%. There is no statistical difference between the activities of the antibodies stored at the different temperatures. It is indicated that the above antibodies all have the excellent stability, and the mutations of the sites have no effect on the stability.

Finally, it should be noted that the above embodiments are only used to illustrate technical schemes of the present disclosure, and not to limit them; although the present disclosure is described in detail with reference to the foregoing embodiments, it should be understood by those of ordinary skill in the art that: it is still possible to modify the technical schemes recorded in the foregoing embodiments, or equivalent replacements are made to some or all of technical features; and these modifications or replacements do not make the essence of the corresponding technical schemes deviate from a scope of the technical schemes of each embodiment of the present disclosure.

### INDUSTRIAL APPLICABILITY

The antibody against the Pan-species-specific antigen Plasmodium Lactate DeHydrogenase (Pan-PLDH) or the binding protein thereof of the present disclosure may bind or recognize the LDH of four types of the plasmodium, therefore it may be used to diagnose the malaria caused by the four types of the plasmodium. Moreover, the detection method for the antibody against the Pan-species-specific antigen Plasmodium Lactate DeHydrogenase (Pan-PLDH) or the binding protein thereof of the present disclosure is used, for example, an immunochromatographic rapid diagnostic reagent method. The method is simple in operation, rapid, intuitive in results, and high in sensitivity and specificity without a complicated device, and is suitable for on-site diagnosis of the malaria.

What is claimed is:

1. An isolated binding protein comprising an antigen binding domain, wherein the antigen binding domain comprises at least one complementarity determining region selected from the following amino acid sequences; or, has at least 80% of sequence identity with the complementarity determining region of the following amino acid sequences and has an affinity of K_{D}≤1.5647×10⁻⁹ mol/L with a pan-species-specific plasmodium lactate dehydrogenase;
a complementarity determining region CDR-VH1 is G-X1-S-F-T-N-Y-X2-M-N, wherein
X1 is S, Y or T, and X2 is W or F;
a complementarity determining region CDR-VH2 is I-X1-P-S-X2-S-E-T-R-X3-N-Q, wherein
X1 is H or N, X2 is E or D, and X3 is I, V or L;
a complementarity determining region CDR-VH3 is A-X1-S-G-X2-F-Y-T-X3-Y-X4-D-Y, wherein,
X1 is K or R, X2 is D or E, X3 is S, Y or T, and X4 is F or W;
a complementarity determining region CDR-VL1 is R-G-X1-G-N-X2-H-N-Y-X3-A, wherein,
X1 is S or T, X2 is I, V or L, and X3 is I or L;
a complementarity determining region CDR-VL2 is N-A-X1-T-X2-A-D, wherein
X1 is R or K, and X2 is I, V or L;
a complementarity determining region CDR-VL3 is Q-X1-F-W-S-X2-Y-T, wherein
X1 is S, Y or T, and X2 is S or T.

2. The isolated binding protein comprising the antigen binding domain as claimed in claim 1, wherein the binding protein comprises at least 3 CDRs; or, the binding protein comprises at least 6 CDRs;
preferably, the binding protein is one of a nano-antibody, F(ab′)₂, Fab′, Fab, Fv, scFv, diabodies, and an antibody minimum recognition unit.

3. The isolated binding protein comprising the antigen binding domain as claimed in claim 1, wherein the binding protein further comprises an antibody constant region sequence;
preferably, the constant region sequence is selected from a sequence of a constant region of any one of IgG1, IgG2, IgG3, IgG4, IgA, IgM, IgE, and IgD;
preferably, the species source of the constant region is cattle, horse, dairy cow, pig, sheep, goat, rat, mouse, dog, cat, rabbit, camel, donkey, deer, mink, chicken, duck, goose, turkey, gamecock or human.

4. An isolated nucleic acid molecule, wherein the nucleic acid molecule is DNA or RNA encoding the binding protein as claimed in claim 1.

5. A vector comprising the nucleic acid molecule as claimed in claim 4.

6. A host cell transformed by the vector as claimed in claim 5.

7. A method for producing the binding protein as claimed in claim 1, comprising the following steps:
culturing a host cell in a culture medium and under a suitable culture condition, and
recovering the binding protein produced from the culture medium or from the cultured host cell;
wherein the host cell is transformed with a vector comprising a nucleic acid molecule,
wherein the nucleic acid molecule encodes the binding protein as claimed in claim 1.

8. (canceled)

9. A method for detecting a pan-species-specific antigen plasmodium lactate dehydrogenase in a test sample, comprising:
a) under a condition sufficient for an antibody/antigen binding reaction to occur, contacting a pan-species-specific antigen plasmodium lactate dehydrogenase antigen with the binding protein as claimed in claim 1 so as to form an immune complex; and
b) detecting the presence of the immune complex, the presence of the immune complex indicates the presence of the pan-species-specific antigen plasmodium lactate dehydrogenase in the test sample.

10. A kit comprising the binding protein as claimed in claim 1.

11. (canceled)

12. The method as claimed in claim 9, wherein the test sample is from a subject, and the presence of the immune complex indicates the presence of the malaria.

13. The method as claimed in claim 9, wherein the method is based on a fluorescence immunoassay technology, a chemiluminescence technology, a colloidal gold immunoassay technology, a radioimmunoassay and/or an enzyme-linked immunoassay technology.

14. The method as claimed in claim 9, wherein the sample is selected from at least one of whole blood, peripheral blood, serum or plasma.

15. The method as claimed in claim 12, wherein the subject is a mammal, preferably a primate, more preferably a human.

16. The method as claimed in claim 12, wherein the malaria is a malaria caused by Plasmodium; preferably, wherein the malaria is selected from a group consisting of Plasmodium vivax; Plasmodium falciparum, Plasmodium malariae, Plasmodium ovale or a combination thereof.

17. (canceled)

18. The isolated binding protein comprising an antigen binding domain as claimed in claim 1, wherein
in the complementarity determining region CDR-VH1, the X2 is W;
in the complementarity determining region CDR-VH2, the X1 is H;
in the complementarity determining region CDR-VH3, the X4 is F;
in the complementarity determining region CDR-VL1, the X3 is L; and
in the complementarity determining region CDR-VL3, the X2 is T;
preferably, in the complementarity determining region CDR-VH1, the X1 is S;
preferably, in the complementarity determining region CDR-VH1, the X1 is Y;
preferably, in the complementarity determining region CDR-VH1, the X1 is T;
preferably, in the complementarity determining region CDR-VH2, the X2 is E;
preferably, in the complementarity determining region CDR-VH2, the X2 is D;
preferably, in the complementarity determining region CDR-VH2, the X3 is I;
preferably, in the complementarity determining region CDR-VH2, the X3 is V;
preferably, in the complementarity determining region CDR-VH2, the X3 is L;
preferably, in the complementarity determining region CDR-VH3, the X1 is K;
preferably, in the complementarity determining region CDR-VH3, the X1 is R;
preferably, in the complementarity determining region CDR-VH3, the X2 is D;
preferably, in the complementarity determining region CDR-VH3, the X2 is E;
preferably, in the complementarity determining region CDR-VH3, the X3 is S;
preferably, in the complementarity determining region CDR-VH3, the X3 is Y;
preferably, in the complementarity determining region CDR-VH3, the X3 is T;
preferably, in the complementarity determining region CDR-VL1, the X1 is S;
preferably, in the complementarity determining region CDR-VL1, the X1 is T;
preferably, in the complementarity determining region CDR-VL1, the X2 is I;
preferably, in the complementarity determining region CDR-VL1, the X2 is V;
preferably, in the complementarity determining region CDR-VL1, the X2 is L;
preferably, in the complementarity determining region CDR-VL2, the X1 is R;
preferably, in the complementarity determining region CDR-VL2, the X1 is K;
preferably, in the complementarity determining region CDR-VL2, the X2 is I;
preferably, in the complementarity determining region CDR-VL2, the X2 is V;
preferably, in the complementarity determining region CDR-VL2, the X2 is L;
preferably, in the complementarity determining region CDR-VL3, the X1 is S;
preferably, in the complementarity determining region CDR-VL3, the X1 is Y;
preferably, in the complementarity determining region CDR-VL3, the X1 is T.

19. The isolated binding protein comprising an antigen binding domain as claimed in claim 1, wherein mutation sites of each complementarity determining region are selected from any one of the following mutation combinations:

| | CDR-VH1 | CDR-VH2 | CDR-VH3 | CDR-VL1 | CDR-VL2 | CDR-VL3 |
|---|---|---|---|---|---|---|
| Site | X1 | X2/X3 | X1/X2/X3 | X1/X2 | X1/X2 | X1 |
| Mutation | S | E/I | K/D/S | S/I | R/I | S |
| combination 1 | | | | | | |
| Mutation | Y | E/L | K/D/Y | T/L | R/V | Y |
| combination 2 | | | | | | |
| Mutation | T | E/V | K/D/T | S/V | R/L | T |
| combination 3 | | | | | | |
| Mutation | T | D/I | K/E/S | T/V | K/I | Y |
| combination 4 | | | | | | |
| Mutation | Y | D/L | K/E/Y | S/I | K/V | T |
| combination 5 | | | | | | |
| Mutation | S | D/V | K/E/T | T/I | K/L | S |
| combination 6 | | | | | | |
| Mutation | T | D/I | R/D/S | T/I | K/L | T |
| combination 7 | | | | | | |
| Mutation | S | D/L | R/D/Y | S/I | K/V | S |
| combination 8 | | | | | | |
| Mutation | Y | D/V | R/D/T | T/V | K/I | Y |
| combination 9 | | | | | | |
| Mutation | S | E/I | R/E/S | S/V | R/L | S |
| combination 10 | | | | | | |
| Mutation | T | E/L | R/E/Y | T/L | R/V | Y |
| combination 11 | | | | | | |
| Mutation | Y | E/V | R/E/T | S/L | R/I | T |
| combination 12 | | | | | | |
| Mutation | Y | D/I | K/D/S | S/L | K/V | Y |
| combination 13 | | | | | | |
| Mutation | T | D/L | K/D/Y | T/L | K/L | T |
| combination 14 | | | | | | |
| Mutation | S | D/V | K/D/T | S/V | K/I | S |
| combination 15 | | | | | | |
| Mutation | S | E/I | K/E/S | T/V | R/V | T |
| combination 16 | | | | | | |
| Mutation | Y | E/L | K/E/Y | S/I | R/L | S |
| combination 17 | | | | | | |
| Mutation | T | E/V | K/E/T | T/I | R/V | T |
| combination 18 | | | | | | |
| Mutation | T | D/I | R/D/S | T/I | K/I | S |
| combination 19 | | | | | | |
| Mutation | Y | D/L | R/D/Y | S/I | K/L | Y |
| combination 20 | | | | | | |
| Mutation | S | D/V | R/D/T | T/V | KV | T |
| combination 21 | | | | | | |
| Mutation | T | E/I | R/E/S | S/V | R/I | Y |
| combination 22 | | | | | | |
| Mutation | S | E/L | R/E/Y | T/L | R/L | T |
| combination 23 | | | | | | |
| Mutation | Y | E/V | R/E/T | S/I | R/V | S |
| combination 24 | | | | | | |
| Mutation | S | D/I | K/D/S | S/L | K/L | T |
| combination 25 | | | | | | |
| Mutation | T | D/L | K/D/Y | T/L | K/I | S |
| combination 26 | | | | | | |
| Mutation | Y | D/V | K/D/T | S/V | K/V | Y |
| combination 27 | | | | | | |
| Mutation | Y | E/I | K/E/S | T/V | R/L | S |
| combination 28 | | | | | | |
| Mutation | T | E/L | K/E/Y | S/I | R/I | Y |
| combination 29 | | | | | | |
| Mutation | S | E/V | K/E/T | T/I | R/V | T |
| combination 30 | | | | | | |
| Mutation | S | D/I | R/D/S | T/I | K/L | Y |
| combination 31 | | | | | | |
| Mutation | Y | D/L | R/D/Y | S/I | K/V | T |
| combination 32 | | | | | | |
| Mutation | T | D/V | R/D/T | T/V | K/I | S |
| combination 33 | | | | | | |
| Mutation | T | E/I | R/E/S | S/V | R/L | T |
| combination 34 | | | | | | |
| Mutation | Y | E/L | R/E/Y | T/L | R/V | S |
| combination 35 | | | | | | |
| Mutation | S | E/V | R/E/T | S/L | R/I | Y |
| combination 36 | | | | | | |
| Mutation | T | D/I | K/D/S | S/L | K/V | S |
| combination 37 | | | | | | |
| Mutation | S | D/L | K/D/Y | T/L | K/I | Y |
| combination 38 | | | | | | |
| Mutation | Y | D/V | K/D/T | S/V | K/L | T |
| combination 39 | | | | | | |
| Mutation | S | E/I | K/E/S | T/V | R/V | Y |
| combination 40 | | | | | | |
| Mutation | T | E/L | K/E/Y | S/I | R/I | T |
| combination 41 | | | | | | |
| Mutation | Y | E/V | K/E/T | T/I | R/L | S |
| combination 42 | | | | | | |
| Mutation | Y | D/I | R/D/S | T/I | K/I | T |
| combination 43 | | | | | | |
| Mutation | T | D/L | R/D/Y | S/I | K/V | S |
| combination 44 | | | | | | |
| Mutation | S | D/V | R/E/S | T/V | K/L | Y |
| combination 45 | | | | | | |
| Mutation | Y | E/I | R/E/Y | S/V | R/I | S |
| combination 46 | | | | | | |
| Mutation | T | E/L | R/E/T | T/L | R/V | Y |
| combination 47 | | | | | | |
| Mutation | T | E/V | K/D/S | S/L | R/L | T |
| combination 48 | | | | | | |
| Mutation | Y | D/I | K/D/Y | S/L | K/L | Y |
| combination 49 | | | | | | |
| Mutation | S | D/L | K/D/T | T/L | K/V | T |
| combination 50 | | | | | | |
| Mutation | T | D/V | K/E/S | S/V | K/I | S |
| combination 51 | | | | | | |
| Mutation | S | E/I | K/E/Y | T/V | R/L | T |
| combination 52 | | | | | | |
| Mutation | Y | E/L | K/E/T | S/I | R/V | S |
| combination 53 | | | | | | |
| Mutation | S | E/V | R/D/S | T/I | R/I | Y |
| combination 54 | | | | | | |
| Mutation | T | D/I | R/D/Y | T/I | K/V | S |
| combination 55 | | | | | | |
| Mutation | Y | D/L | R/D/T | S/I | K/I | Y |
| combination 56 | | | | | | |

20. The isolated binding protein comprising the antigen binding domain as claimed in claim 1, wherein the binding protein is labeled with an indicator for displaying signal intensity.

21. The isolated binding protein comprising the antigen binding domain as claimed in claim 1, wherein the binding protein comprises sequences of light chain framework regions FR-L1, FR-L2, FR-L3 and FR-L4 successively shown in SEQ ID NO 1-4, and/or sequences of heavy chain framework regions FR-H1, FR-H2, FR-H3 and FR-H4 successively shown in SEQ ID NO: 5-8.

22. The isolated binding protein comprising the antigen binding domain as claimed in claim 1, wherein the constant region is derived from the mouse;
a light chain constant region sequence is shown in SEQ ID NO: 9; and
a heavy chain constant region sequence is shown in SEQ ID NO:10.

23. The method as claimed in claim 9, wherein,
in the step a), the immune complex further comprises a second antibody, and the second antibody binds to the binding protein; or
in the step a), the immune complex further comprises a second antibody, and the second antibody binds to the pan-species-specific antigen plasmodium lactate dehydrogenase.


SEQUENCE LISTING (28 sequences)

SEQ ID NO: 1 - protein, 23 aa, Mus musculus
DIQLTQSPASLSASVGETVTITC

SEQ ID NO: 2 - protein, 15 aa, Mus musculus
WYQQKQGKSPQLLVY

SEQ ID NO: 3 - protein, 32 aa, Mus musculus
GVPSRFSGSGSGTQYSLKINSLQPEDFGSYYC

SEQ ID NO: 4 - protein, 10 aa, Mus musculus
FGGGTKLEIK

SEQ ID NO: 5 - protein, 25 aa, Mus musculus
QVQLQQPGAELVRPGASVKLSCKAS

SEQ ID NO: 6 - protein, 15 aa, Mus musculus
WVKQRPGQGLEWIGM

SEQ ID NO: 7 - protein, 34 aa, Mus musculus
KFKDKATLTVDKSSSTAYMQLSSLTAEDSAVYYC

SEQ ID NO: 8 - protein, 11 aa, Mus musculus
WGQGTTLTVSS

SEQ ID NO: 9 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 335 aa, Mus musculus
AKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 11 - protein, 213 aa, Mus musculus
DIQLTQSPASLSASVGETVTITCRGSGNLHNYIAWYQQKQGKSPQLLVYNARTIADGVPSRFSGSGSGTQYSLKINSLQPEDFGSYYCQSFWSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 455 aa, Mus musculus
QVQLQQPGAELVRPGASVKLSCKASGSSFTNYFMNWVKQRPGQGLEWIGMINPSESETRINQKFKDKATLTVDKSSSTAYMQLSSLTAEDSAVYYCAKSGDFYTSYWDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 13 - protein, 10 aa, Artificial Sequence
  mutated CDR-VH1
  MISC_FEATURE (2)..(2): Xaa is S, Y or T
  MISC_FEATURE (8)..(8): Xaa is W or F
GXSFTYNXMN

SEQ ID NO: 14 - protein, 12 aa, Artificial Sequence
  mutated CDR-VH2
  MISC_FEATURE (2)..(2): Xaa is H or N
  MISC_FEATURE (5)..(5): Xaa is D or E
  MISC_FEATURE (10)..(10): Xaa is I, V or L
IXPSXSETRXNQ

SEQ ID NO: 15 - protein, 13 aa, Artificial Sequence
  mutated CDR-VH3
  MISC_FEATURE (2)..(2): Xaa is K or R
  MISC_FEATURE (5)..(5): Xaa is D or E
  MISC_FEATURE (9)..(9): Xaa is S, T or Y
  MISC_FEATURE (11)..(11): Xaa is F or W
AXSGXFYTXYXDY

SEQ ID NO: 16 - protein, 11 aa, Artificial Sequence
  mutated CDR-VL1
  MISC_FEATURE (3)..(3): Xaa is S or T
  MISC_FEATURE (6)..(6): Xaa is I, V or L
  MISC_FEATURE (10)..(10): Xaa is I or L
RGXGNXHNYXA

SEQ ID NO: 17 - protein, 7 aa, Artificial Sequence
  mutated CDR-VL2
  MISC_FEATURE (3)..(3): Xaa is R or K
  MISC_FEATURE (5)..(5): Xaa is V, I or L
NAXTXAD

SEQ ID NO: 18 - protein, 8 aa, Artificial Sequence
  mutated CDR-VL3
  MISC_FEATURE (2)..(2): Xaa is S, Y or T
  MISC_FEATURE (6)..(6): Xaa is  S or T
QXFWSXYT

SEQ ID NO: 19 - DNA, 25 bp, Artificial Sequence
  SMARTER II A oligonucleotide from a commerical kit
  misc_feature (25)..(25): XXXXX at the 3′-end
aagcagtggtatcaacgcagagtac

SEQ ID NO: 20 - DNA, 27 bp, Artificial Sequence
  5′-RACE CDS primer
  misc_feature (26)..(26): v is a, c or g
  misc_feature (27)..(27): n is a, t, c or g
tttttttttttttttttttttttttvn

SEQ ID NO: 21 - DNA, 45 bp, Artificial Sequence
  Universal primer A mixture
ctaatacgactcactatagggcaagcagtggtatcaacgcagagt

SEQ ID NO: 22 - DNA, 23 bp, Artificial Sequence
  Nested universal primer A
aagcagtggtatcaacgcagagt

SEQ ID NO: 23 - DNA, 26 bp, Artificial Sequence
  Synthesized mkR
cgcctaacactcattcctgttgaagc

SEQ ID NO: 24 - DNA, 22 bp, Artificial Sequence
  Synthesized mHR
ccgctcatttacccggagaccg

SEQ ID NO: 25 - DNA, 46 bp, Artificial Sequence
  Synthesized Pan-9G7-HF
cccaagcttgccgccaccatgagtgtgctcactcaggtcctggggt

SEQ ID NO: 26 - DNA, 40 bp, Artificial Sequence
  Synthesized Pan-9G7-HR
ggggaattctcatttacccggagaccgggagatggtcttc

SEQ ID NO: 27 - DNA, 45 bp, Artificial Sequence
  Synthesized Pan-9G7-LF
cccaagcttgccgccaccatgaagtcacagacccaggtcttcgta

SEQ ID NO: 28 - DNA, 43 bp, Artificial Sequence
  Synthesize Pan-9G7-LR
cccgaattctcaacactcattcctgttgaagctcttgacgatg